(19) United States
(12) Patent Application Publication
(10) Pub. No.: US 2008/0168988 A1
(43) Pub. Date: Jul. 17, 2008

(54) Medication-saving device for inhalation therapy

(76) Inventor: Hsueh-Yu Lu, Taoyuan (TW)

Correspondence Address: TROXELL LAW OFFICE PLLC, Suite 1404, 5205 Leesburg Pike, Falls Church, VA, 22041, US

(21) Appl. No.: 11/652,670

(22) Filed: Jan. 12, 2007

(51) Int. Cl. A61M 15/00

(52) U.S. Cl. 128/203.15

(57) ABSTRACT

A medication-saving device for inhalation therapy comprises a tube joint mounted under a nebulizer bottle, wherein the tube joint has an opening. A control button is inserted into the opening. The high pressure air is prevented from leakage and enabled to siphon the medicinal liquid for generating the nebulization effect by push-pressing the control button. In addition, the control button can be shifted backward to form a gap for draining out some of the high pressure air so that the medicinal liquid can not be siphoned by the high pressure air. Moreover, the patient is enabled to decide to supply the medicine or not by controlling the control button. As a result, the waste of medicinal liquid, which is formerly sprayed out ceaselessly, can be thus avoided.

### FIELD OF THE INVENTION

The present invention relates to a medication-saving device for inhalation therapy, and more particularly to a medication-saving device for inhalation therapy capable of coupling with the existing commercial product.

### BACKGROUND OF THE INVENTION

The therapeutic inhalator is a therapeutic tool for curing the respiratory diseases in the daily life. Particularly the patients with bronchial asthma or chronic diseases usually suffer from breathing difficulties caused by emotion change, chemical irritants, or allergens or climate change. At this point, timely injection or eat of anti-inflammatory drugs or bronchiectasis agent or spray treatment are essential for these patients. As the effects of injection or oral drugs are slow, the nebulization-type therapeutic inhalators have been widely adopted.

Above all, a mechanical nebulizer is disclosed for generating high pressure air flow to nebulize the medicinal liquid for spraying out the fine medicinal liquid particles so that the medicinal liquid particles can be guided to reach the lesions of the patients through inhalation. The existing therapeutic inhalators for curing the respiratory diseases are only pure therapeutic tools, wherein the medicinal liquid is sprayed out ceaselessly when the- patients change breath or stop inhaling transiently.

### SUMMARY OF THE INVENTION

A first object of the present invention is to provide medication-saving device for inhalation therapy to enable the patient to decide to supply the medicine or not by controlling the control button and to avoid the waste of the medicinal liquid, which is sprayed out ceaselessly when the supply of medicinal liquid is not needed transiently.

A second object of the present invention is to provide a simple and easy medication-saving device capable of coupling with the existing product without modifying the existing structure of nebulizer bottle.

A third object of the present invention is to utilize the control button mounted on the tube joint to control the air supply status easily for further nebulizing the medicinal liquid or not so as to bring the effect of medication into full play.

In order to achieve the aforesaid objects, a medication-saving device for inhalation therapy, which is comprised of a tube joint mounted under a nebulizer bottle, is disclosed. A control button is mounted on the tube joint to communicate with an air-supply tunnel. As a result, the supply amount of high pressure inlet air can be controlled for controlling the nebulization effect of the medicinal liquid by controlling the control button.

### BRIEF DESCRIPTION OF THE DRAWINGS

FIG. 1 is an elevational diagram of the present invention.

FIG. 2 is a cross-sectional view showing the assembled structure of the present invention.

FIG. 3 is a diagram showing the nebulization process of the medicinal liquid in accordance a preferred embodiment of the present invention.

FIG. 4 is an enlarged view showing the encircled region A of FIG. 2.

FIG. 5 is a schematic view showing the usage status of the structure shown in FIG. 4.

FIG. 6 is a cross-sectional view showing the control button of the present invention that has an elastic device attached thereon.

FIG. 7 is a schematic view showing a first usage status of the control button in accordance with another preferred embodiment of the present invention.

FIG. 8 is a schematic view showing a second usage status of the control button in accordance with the other preferred embodiment of the present invention.

FIG. 9 is a schematic view showing a first usage status of the control button in accordance with a further preferred embodiment of the present invention.

FIG. 10 is a schematic view showing a second usage status of the control button in accordance with the further preferred embodiment of the present invention.

FIG. 11 is a schematic view showing a first usage status of using the finger to open the air outlet in accordance with the present invention.

FIG. 12 is a schematic view showing a second usage status of using the finger to close the air outlet in accordance with the present invention.

### DETAILED DESCRIPTION OF THE PREFERRED EMBODIMENT

It will be understood that the present disclosure is for the purpose of illustration only and the invention extends to modifications, variations and improvements thereto. Thus, the breadth and scope of a preferred embodiment should not be limited by any of the exemplary embodiments, but should be defined only in accordance with the following claims and their equivalents.

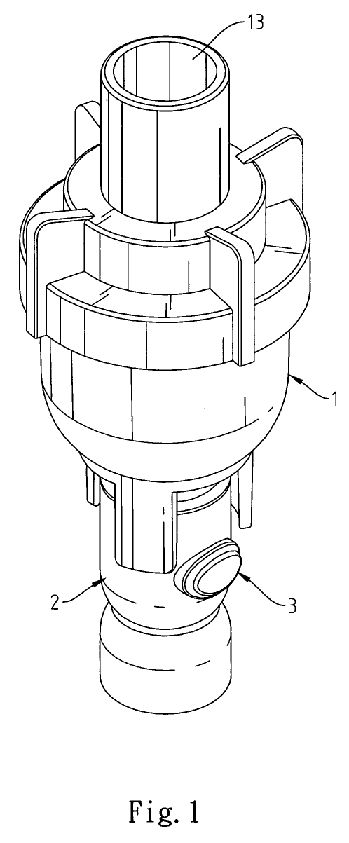

Referring to FIG. 1 through FIG. 3, a medication-saving device for inhalation therapy of the present invention generally comprises a tube joint 2 mounted under a nebulizer bottle 1.

The nebulizer bottle 1 has a storage tank 11 for storing medicinal liquid 4, wherein the bottom of the storage tank 11 is communicated with an air-intake duct 12. This air-intake duct 12 is extended to the top of the storage tank 11. In addition, a nebulizing means 14 is sleeved onto the outside of the air-intake duct 12 inside the storage tank 11 so as to form a tunnel 15 between the air-intake duct 12 and the nebulizing means 14 so that the medicinal liquid 4 can be siphoned upward rapidly via the tunnel 15 by the negative pressure formed by the high pressure gas that enters the air-intake duct 12. As a result, the nebulized medicinal liquid 4 can be sprayed out via a shower nozzle 13.

The air-intake duct 12, which is mounted on the lower portion of the nebulizer bottle 1, is coupled with the tube joint 2 so that the tube joint 2 can control the supply of high pressure inlet air. If the pressure of the inlet air is less than a predetermined pressure value, the medicinal liquid 4 stored in the nebulizer bottle 1 cannot be driven to form mists, wherein the present invention achieves the purpose of saving medication by utilizing this principle.

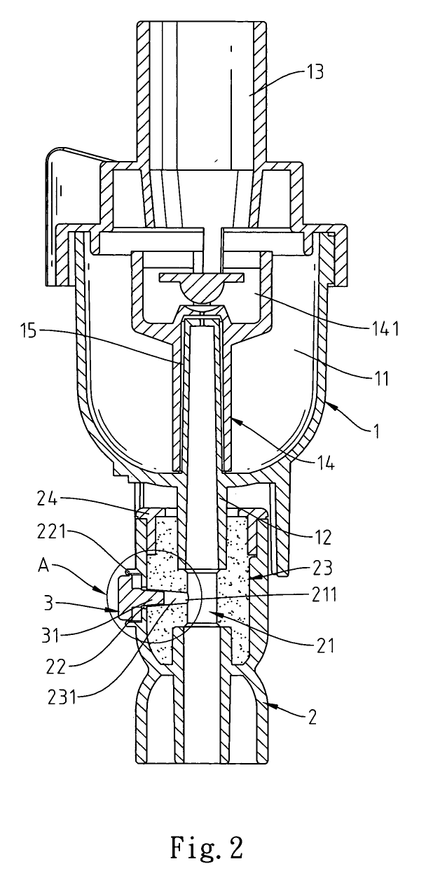

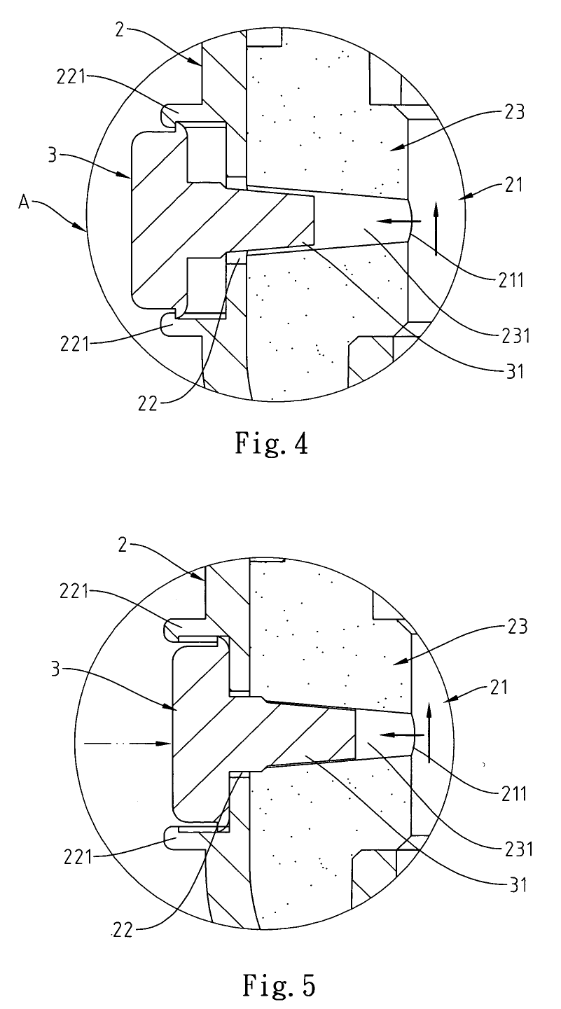

Referring to FIG. 2 through FIG. 5, the structure of the tube joint 2 is shown. The inside of the tube joint 2 is communicated with an air-supply tunnel 21. An anti-leakage soft means 23 is plugged between the tube joint 2 and the air-supply tunnel 21. The anti-leakage soft means 23 has a communicating tunnel 231 corresponding to a gas hole 211 of the air-supply tunnel 21. Because the communicating tunnel 231 is communicated with the gas hole 211 to further communicate with the air-supply tunnel 21, the high pressure air that enters the air-supply tunnel 21 can be drained out via the gas hole 211.

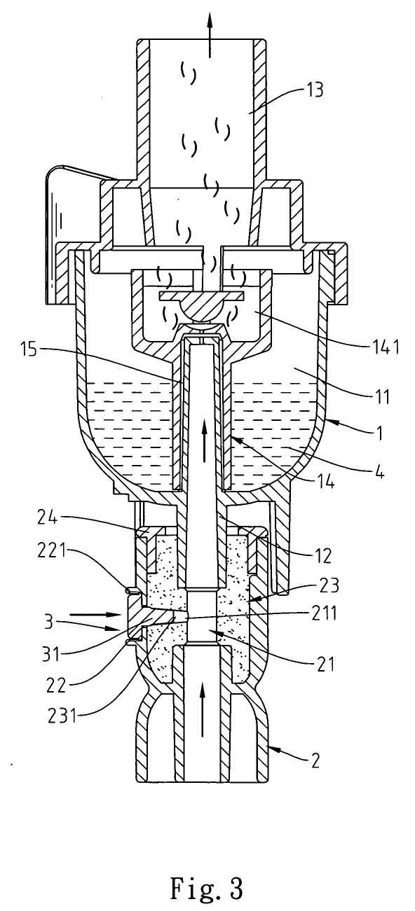

Referring further to FIG. 3 through FIG. 5, a medication-saving device for inhalation therapy is formed by utilizing the existing structure of the nebulizer bottle 1. The tube joint 2 can be attached to the nebulizer bottle 1 easily without modifying the structure of the nebulizer bottle 1. The high pressure air can be transported via the tube joint 2, and coupled with the medicinal liquid 4 so as to generate the nebulization effect.

The tube joint 2 is designed for coupling the high pressure air with the medicinal liquid 4 so as to generate the nebulization effect. The tube joint 2 has an opening 22 that communicates with the gas hole 211 and the communicating tunnel 231. A control button 3 is inserted into the opening 22, wherein the control button 3 has a pillar 31 for insertion into the communicating tunnel 231. The pillar 31 of the control button 3 can be tightly coupled with communicating tunnel 231 by push-pressing the control button 3 to prevent the high pressure air from leakage and to allow the high pressure air to siphon the medicinal liquid 4 for generating the nebulization effect. In addition, the control button 3 can be shifted backward to form a gap for leaking out some of the high pressure air so that the medicinal liquid 4 can not be siphoned by the high pressure air. Accordingly, the patient is enabled to decide to supply the medicine or not by controlling the control button 3. As a result, the waste of the medicinal liquid, which is formerly sprayed out ceaselessly, can be thus avoided.

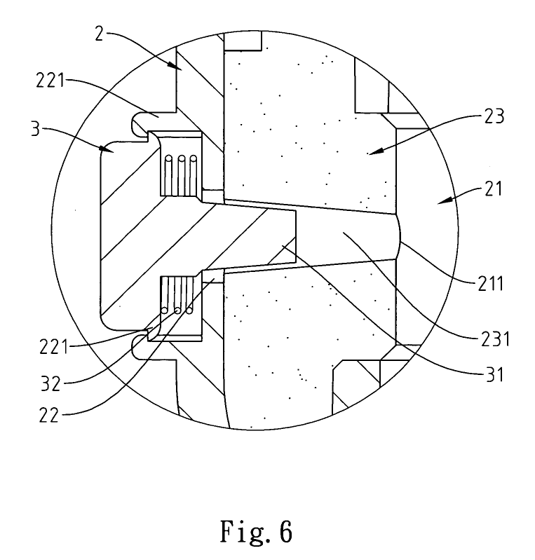

Referring to FIG. 6, the control button 3 is shown. The pillar 31 of the control button 3 is sleeved into an elastic device 32. The elastic device 32 is held in the opening 22 of the tube joint 2 to enable the control button 3 to restore the location after press automatically. In addition, the control button 3 has a hook 221 in the opening 22 to prevent the control button 3 from detachment.

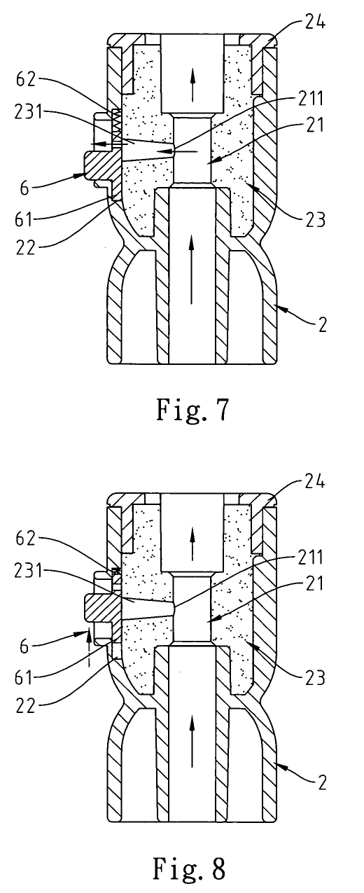

Referring further to FIG. 7 and FIG. 8, another preferred embodiment of the present invention is shown. A main body 61 of a control button 6 is located in the opening 22 of the tube joint 2. An elastic device 62 is attached to one end of the main body 61 so that the control button 6 is upward and downward shiftable. By shifting the control button 6, the air-supply tunnel 21 of the tube joint 2 can be opened to leak out the high pressure air via the opening 22, the gas hole 211 and the communicating tunnel 231. In addition, when the opening 22 is closed by the control button 6, the high pressure air can be guided to pass the nebulizer bottle 1 so as to nebulize the medicinal liquid 4 by siphon action.

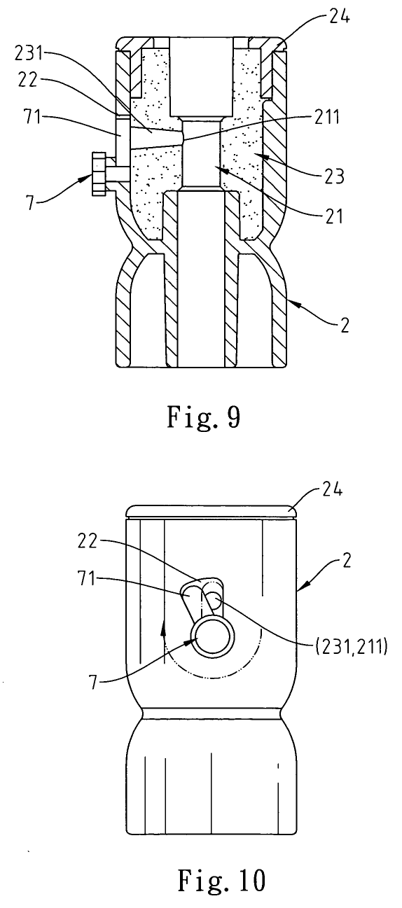

Referring further to FIG. 9 and FIG. 10, a control button 7 is angularly shiftable in the opening 22 of the tube joint 2. The control button 7 has a shelter 71. The gas hole 211 and the communicating tunnel 231 that communicate with the opening 22 can be exactly opened or closed by rotating the shelter 71 of the control button 7. As a result, the supply amount of high pressure inlet air can be controlled for further controlling the nebulization effect of the medicinal liquid by rotating the control button 7.

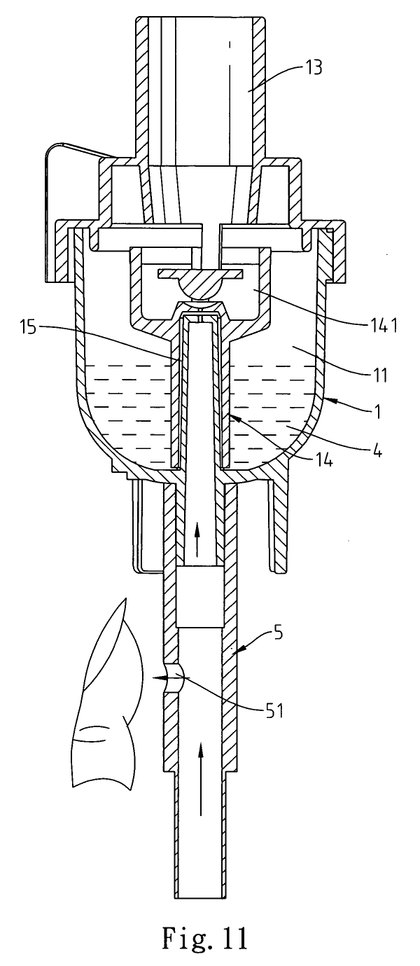

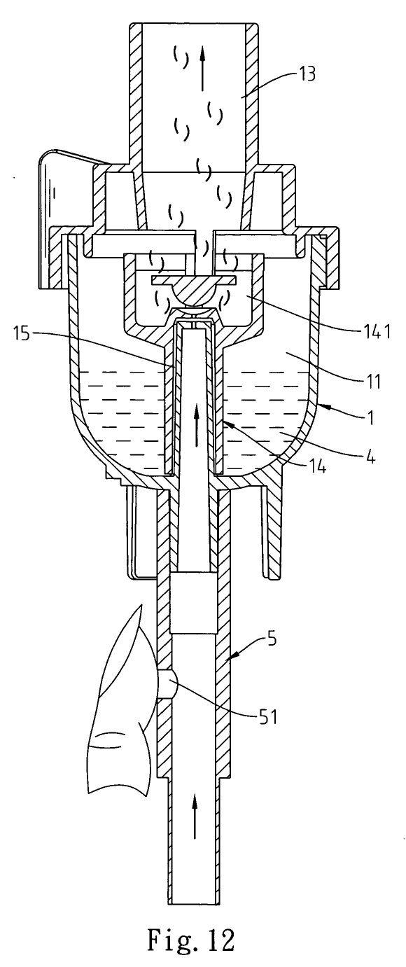

In addition, as shown in FIG. 11 and FIG. 12, the finger is utilized to open or close an air outlet of the medication-saving device, wherein the nebulizer bottle 1 can be coupled with a tube joint 5 easily. The tube joint 5 has an air outlet 51. The high pressure air can be guided to pass the nebulizer bottle 1 for nebulizing the medicinal liquid 4 by utilizing the finger to open or close the air outlet 51.

What the invention claimed is:

1. A medication-saving device for inhalation therapy comprising:
a nebulizer bottle; and
a tube joint mounted under said nebulizer bottle, said pipe joint having an air-supply tunnel for communication with the inside thereof, an opening for communication with said air-supply tunnel, and a control button for coupling with said opening of said tube joint so that a supply amount of high pressure air can be controlled by operating said control button.

2. A medication-saving device for inhalation therapy of claim 1, wherein an anti-leakage soft means is plugged between said tube joint and said air-supply tunnel.

3. A medication-saving device for inhalation therapy of claim 1, wherein an elastic device is mounted on said control button in said opening.

4. A medication-saving device for inhalation therapy of claim 1, wherein said control button has a shelter to open or close a gas hole and a communicating tunnel that communicate with said opening by rotating said shelter of said control button.
